(19) United States
(12) Patent Application Publication
(10) Pub. No.: US 2003/0212442 A1
(43) Pub. Date: Nov. 13, 2003

(54) Low level light therapy for the treatment of myocardial infarction

(76) Inventor: Jackson Streeter, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/328,153

(22) Filed: Dec. 23, 2002

Related U.S. Application Data
(60) Provisional application No. 60/369,260, filed on Apr. 2, 2002.
(60) Provisional application No. 60/345,177, filed on Dec. 21, 2001.

(51) Int. Cl. A61N 1/00

(52) U.S. Cl. 607/88

(57) ABSTRACT

Therapeutic methods for the treatment of myocardial infarction are described, the methods including delivering a myocardial protective effective amount of light energy having a wavelength in the visible to near-infrared wavelength range to that area of the myocardium containing the area of primary infarct. A myocardial protective effective amount of light energy is a selected or predetermined power density (mW/cm²) at the level of the myocardial tissue being treated, and is determined by determining a surface power density of the light energy sufficient to deliver the selected power density of light energy to the myocardial tissue taking into account factors that attenuate the light energy as it travels from the skin surface to the myocardial tissue being treated.

### BACKGROUND OF THE INVENTION

1. Field of the Invention

The present invention relates in general to therapeutic methods for the treatment of myocardial infarction, and more particularly to novel methods for reducing the size of myocardial infarction using light therapy

2. Description of the Related Art

Myocardial ischemia refers to the condition of oxygen deprivation in heart muscle (“myocardium”) that is produced by some imbalance in the myocardial oxygen supply-demand relationship. Myocardial infarction (“MI”), also known as “heart attack”, refers to the death of cells in an area of heart muscle as a result of oxygen deprivation due to obstruction of the blood supply, typically due to occlusion of one or more coronary arteries or branches. Occlusion usually stems from clots that form upon the sudden rupture of an atheromatous plaque through the sublayers of a blood vessel, or when the narrow, roughened inner lining of a sclerosed artery leads to complete thrombosis. Approximately 1.5 million myocardial infarctions (MIs) occur annually, and nearly 500,000 deaths result from ischemic heart disease. The United States alone loses billions of dollars annually to medical care and lost productivity due to cardiovascular disease including myocardial infarction.

Treatment after MI depends on the extent to which the cells have been deprived of oxygen. Complete oxygen deprivation produces a zone of infarction in which cells die and the tissue becomes necrotic, with irretrievable loss of function. However, immediately surrounding the area of infarction is a less seriously damaged region of tissue, the zone of ischemia, in which cells have not been irretrievably damaged by complete lack of oxygen but instead are merely weakened and at risk of dying. If adequate collateral circulation develops, the extended zone may regain function within 2 to 3 weeks. The zone of infarction and the zone of ischemia, are both identifiable using standard diagnostic techniques such as electrocardiography, echocardiography and radionuclide testing.

Therapeutic strategies in treating MI are directed at reducing the final extent of the infarcted region by preserving viable tissue and if possible retrieving surviving but at-risk cells. Known treatment methods for myocardial infarction include surgical interventions and pharmacologic treatments. A combination of therapeutic approaches is sometimes advisable. Selection of the appropriate therapy depends on a number of factors, including the degree of coronary artery occlusion, the extent of existing damage if any, and fitness of the patient surgery. Surgical interventions include coronary artery bypass surgery and percutaneous coronary procedures such as angioplasty, artherectomy and endarterectomy. Pharmacologic agents for treating MI include inhibitors of angiotensin converting enzyme (ACE) such as captopril, quinapril and ramipril, thrombolytic agents including aspirin, streptokinase, t-PA and anistreplase, β-adrenergic anatagonists, Ca⁺⁺ channel blockers, and organic nitrates such as nitroglycerin. However, surgical interventions are invasive and can increase the risk of stroke, and pharmacologic agents carry the risk of eliciting serious adverse side effects and immune responses.

High energy laser radiation is now well accepted as a surgical tool for cutting, cauterizing, and ablating biological tissue. High energy lasers are now routinely used for vaporizing superficial skin lesions and, and to make deep cuts. For a laser to be suitable for use as a surgical laser, it must provide laser energy at a power sufficient to heat tissue to temperatures over 50° C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in desirable biostimulatory effects while leaving tissue undamaged. In rat models of myocardial infarction and ischemia-reperfusion injury, low energy laser irradiation reduces infarct size and left ventricular dilation, and enhances angiogenesis in the myocardium. (Yaakobi et al., J Appl. Physiol. 90, 2411-19 (2001)). Low level laser therapy has been described for treating pain, including headache and muscle pain, and inflammation.

While low level laser therapy has been described for certain limited applications, known low level laser therapy methods are circumscribed by setting selected parameters within specified limits. For example, known methods include setting the power output of the laser source at very low levels of 5 mW to 70 mW, low dosages at about 1-10 Joule/cm², and time periods of application of the laser energy at twenty seconds to minutes. However, other parameters can be varied in the use of low level laser therapy. In particular, known low level laser therapy methods have not accounted for other factors that contribute to the photon density that actually is delivered to tissue and may play key roles in the efficacy of low level laser therapy.

Against the background, a high level of interest remains in finding new and improved therapeutic methods for the treatment of myocardial infarction. In particular, a need remains for relatively inexpensive and non-invasive approaches to treating myocardial infarction that also avoid the limitations of drug therapy.

### SUMMARY OF THE INVENTION

The low level light therapy method for the treatment of myocardial infarction is based in part on the new and surprising discovery that power density (i.e. power per unit area) of the light energy as applied to tissue, and not power or dosage of light energy per se, appears to be an important factor in the relative efficacy of low level light therapy, and particularly with respect to treating and saving surviving but endangered myocardial cells in a zone of ischemia surrounding the primary infarct after a myocardial infarction.

In a broad aspect, methods directed toward the treatment of myocardial infarction in a subject in need of such treatment include delivering a myocardial protective effective amount of light energy having a wavelength in the visible to near-infrared wavelength range to an area of the myocardium of the subject that includes the area of infarct, wherein delivering the myocardial protective effective amount of light energy includes delivering a particular power density of light energy to the area of the myocardium.

Preferred methods further encompass placing a light source in contact with a region of skin adjacent the area of the myocardium that includes the area of infarct to deliver the myocardial protective effective amount of light energy to the area of the myocardium by delivering a selected power density. In addition, to deliver the selected or predetermined power density to the area of the myocardium, the methods encompass determining a surface power density of the light energy sufficient for the light energy to penetrate the skin and any tissue interposed between the skin and the area of myocardium being treated. The determination of the surface power density, which is relatively higher than the power density to be delivered to the myocardial area being treated, takes into account factors that attenuate power density as it travels through tissue, including skin pigmentation, and location of the myocardial area being treated, particularly the distance of the myocardial area from the skin surface where the light energy is applied.

Additional preferred embodiments of the foregoing methods may include one or more. of the following: the selected power density is a power density selected from the range of about 0.01 mW/cm² to about 150 mW/cm²; the light energy has a wavelength of about 780 nm to about 840 nm; and the light is delivered in pulses at a frequency of about 1 Hz to about 1 kHz.

Preferred methods may further encompass selecting a dosage and power of the laser energy sufficient to deliver the predetermined power density of laser energy to the myocardium by selecting the dosage and power of the laser sufficient for the laser energy to penetrate any body tissue, for example a thickness of skin and other bodily tissue such as fat, bone, lung tissue, and muscle that is interposed between the heart and the skin surface adjacent the heart and/or sufficient for the laser energy to traverse the distance between the heart and the skin surface adjacent the heart.

### BRIEF DESCRIPTION OF THE DRAWINGS

FIG. 1 is a perspective view of a first embodiment of a light therapy device; and

FIG. 2 is a block diagram of a control circuit for the light therapy device, according to one embodiment of the invention.

### DETAILED DESCRIPTION OF THE PREFERRED EMBODIMENT

The lower level light therapy methods for the treatment of myocardial infarction described herein are practiced and described using, for example, a low level light therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference together with the references contained therein.

A suitable apparatus for the methods to prevent or retard rejection of a transplanted organ is a low-level light apparatus including a handheld probe for delivering the light energy. The probe includes a light source of light energy having a wavelength in the visible to near-infrared wavelength range, i.e. from about 630 nm to about 904 nm. In one embodiment, the probe includes a single laser diode that provides about 25 mW to about 500 mW of total power output, or multiple laser diodes that together are capable of providing at least about 25 mW to about 500 mW of total power output. In other embodiments, the power provided may be more or less than these stated values. The actual power output is preferably variable using a control unit electronically coupled to the probe, so that the power of the light energy emitted can be adjusted in accordance with required power density calculations as described below. In one embodiment, the diodes used are continuously emitting GaAIAs laser diodes having a wavelength of about 830 nm.

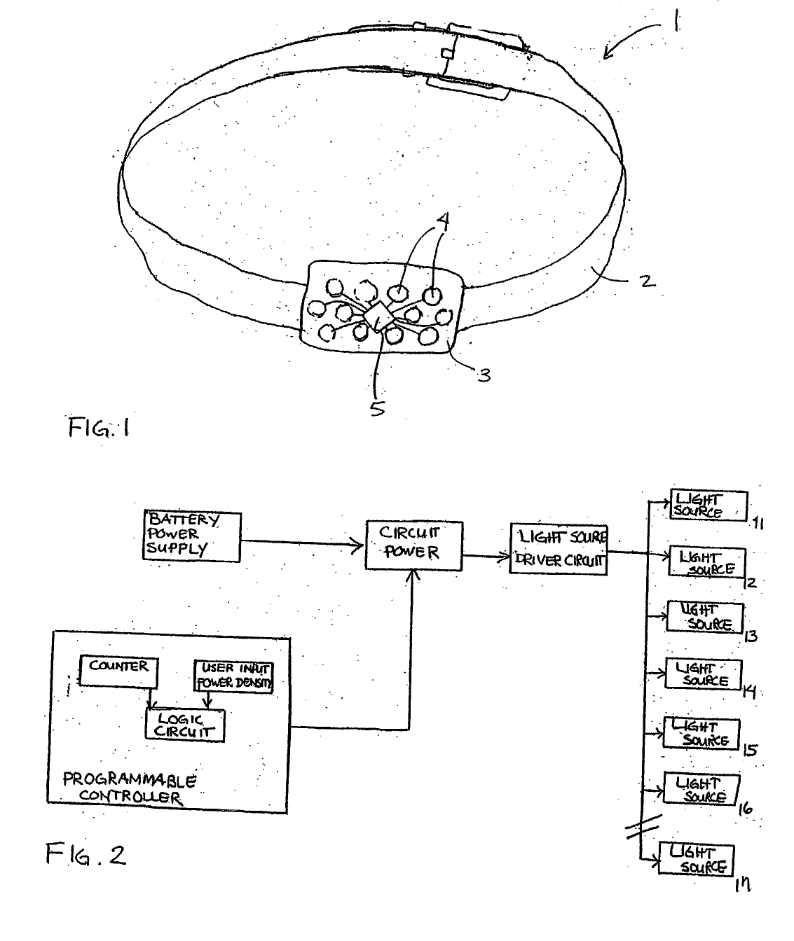

Another suitable light therapy apparatus is that illustrated in FIG. 1. This apparatus is especially preferred for methods in which the light energy is delivered through the skin. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve the predetermined subsurface power density selected by the programmable controller. It is presently believed that tissue will be most effectively treated using subsurface power densities of light of at least about 0.01 mW/cm² and up to about 150 mW/cm², including about 0.05, 0.1, 0.5, 1, 5, 10, 15, 20, 30, 40, 50, 60, 70, 80, 90, 100, 110, 120, 130, and 140 mW/cm². In one embodiment, subsurface power densities of about 10 mW/cm² to about 90 mW/cm² are used. To attain subsurface power densities within these stated ranges, taking into account attenuation of the energy as it travels through body tissue and fluids from the surface to the target tissue, surface power densities of from about 10 mW/cm² to about 500 mW/cm² will typically be required, but also up to about 1000 mW/cm². To achieve such surface power densities, preferred light energy sources, or light energy sources in combination, are capable of emitting light energy having a total power output of at least about 25 mW to about 500 mW, including about 30, 50, 75, 100, 150, 200, 250, 300, and 400 mW, but may also be as high as about 1000 mW. It is believed that the subsurface power densities of at least about 10 mW/cm² and up to about 150 mW/cm² in terms of the power density of energy that reaches the subsurface tissue are especially effective at producing the desired biostimulative effects on tissue being treated.

The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly around the chest. The precise configuration of the strap is subject only to the limitation that the strap is capable of maintaining the light energy sources in a select position relative to the particular area of the body or tissue being treated. In an alternative embodiment, a strap is not used and instead the light source or sources are incorporated into or attachable onto a piece of fabric which fits securely over the target body portion thereby holding the light source or sources in proximity to the patient's body for treatment. The fabric used is preferably a stretchable fabric or mesh comprising materials such as Lycra or nylon. The light source or sources are preferably removably attached to the fabric so that they may be placed in the position needed for treatment.

In the exemplary embodiment illustrated in FIG. 1, a light therapy device includes a flexible strap and securing means such as mating Velcro strips configured to secure the device around the body of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are preferably secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body. Furthermore, the device may be provided without a strap and placed over the area of treatment either with or without additional securement.

FIG. 2 is a block diagram of a control circuit according to one. embodiment of the light therapy device. The programmable controller is configured to select a predetermined surface power density of the light energy sufficient to deliver a predetermined subsurface power density, preferably about 0.01 mW/cm² to about 150 mW/cm², including about 10 mW/cm² to about 100 mW/cm² to the target area. The actual total power output if the light energy sources is variable using the programmable controller so that the power of the light energy emitted can be adjusted in accordance with required surface power energy calculations as described below.

The methods described herein are based in part on the surprising finding that delivering low level light energy within a select range of power density (i.e. light intensity or power per unit area, in mW/cm²) appears to be an important factor for producing therapeutically beneficial effects with low level light energy as applied to heart tissue. Without being bound by theory, it is believed that independently of the power and dosage of the light energy used, light energy delivered within the specified range of power densities provides a biostimulative effect on the intracellular environment, such that proper function is returned to previously non-functioning or poorly functioning mitochondria in at-risk myocardial cells.

The term “myocardial degeneration” refers to the process of cell destruction in the myocardium resulting from primary destructive events such as myocardial infarction, and also secondary, delayed and progressive destructive mechanisms that are invoked by cells due to the occurrence of the primary destructive even. Primary destructive events include myocardial infarction, but also include other diseases and conditions such as physical trauma that may lead to myocardial ischemia. Secondary destructive mechanisms include any mechanism that leads. to the generation and release of cytotoxic molecules, including apoptosis, depletion of cellular energy stores because of changes in mitochondrial membrane permeability, reperfusion injury, and activity of cytokines and inflammation. Both primary and secondary mechanisms contribute to forming a “zone of danger” for myocardial cells, wherein the myocardial cells in the zone have at least temporarily survived the primary destructive event, but are at risk of dying due to processes having delayed effect.

The term “myocardial protection” refers to a therapeutic strategy for slowing or preventing the otherwise irreversible loss of myocardium due to myocardial degeneration after a primary destructive event, whether the degeneration loss is due to disease mechanisms associated with the primary destructive event or due to secondary destructive mechanisms.

The term “myocardial protective effective” as used herein refers to a characteristics of an amount of light energy, wherein the amount if a power density of the light energy measured in mW/cm².. The amount of light energy achieves the goal of preventing, avoiding, reducing or eliminating myocardial degeneration.

In preferred embodiments, treatment parameters include the following. Preferred power densities of light at the level of the target cells are at least about 0.01 mW/cm² to about 150 mW/cm², including about 0.05, 0.1, 0.5, 1, 5, 10, 15, 20, 30, 40, 50, 60, 70, 80, 90, 100, 110, 120, 130, and 140 mW/cm². In some embodiments, higher power densities can be used. To attain subsurface power densities within this preferred range in in vivo methods, one must take into account attenuation of the energy as it travels through body tissue and fluids from the surface to the target tissue, such that surface power densities of from about 25 mW/cm² to about 500 mW/cm² will typically be used, but also possibly to about 1000 mW/cm² or more. To achieve desired power densities, preferred light energy sources, or light energy sources in combination, are capable of emitting light energy having a total power output of at least about 1 mW to about 500 mW, including about 5, 10, 15, 20, 30, 50, 75, 100, 150, 200, 250, 300, and 400 mW, but may also be up to as high as about 1000 mW or below 1 mW. Preferably the light energy used for treatment has a wavelength in the visible to near-infrared wavelength range, i.e., from about 630 to about 904 nm, preferably about 780 nm to about 840 nm, including about 790, 800, 810, 820, and 830 nm.

In preferred embodiments, the light source used in the light therapy is a coherent source (i.e. a laser), and/or the light is substantially monochromatic (i.e. one wavelength or a very narrow band of wavelengths).

In preferred embodiments, the treatment proceeds continuously for a period of about 30 seconds to about 4 hours, including about 10 minutes, 20 min., 30 min., 45 min., 1 hour, 2 hrs., and 3 hrs. Treatment times outside of these ranges are also within the scope of the invention, and may be performed as deemed necessary for effective treatment. The treatment may be terminated after one treatment period, or the treatment may be repeated one or more times, with anywhere from a few hours to a few days passing between treatments. The length of treatment time and frequency of treatment periods can be varied as needed to achieve the desired result.

During the treatment, the light energy may be continuously provided, or it may be pulsed. If the light is pulsed, the pulses are preferably at least about 10 ns long, including about 100 ns, 1 ms, 10 ms, and 100 ms, and occur at a frequency of up to about 1 kHz, including about 1 Hz, 10 Hz, 50 Hz, 100 Hz, 250 Hz, 500 Hz, and 750 Hz.

Generally, light energy suitable for practicing the methods includes light energy in the visible to near-infrared wavelength range, i.e. wavelengths in the range of about 630 nm to about 904 nm. In an exemplary embodiment, the light energy has a wavelength of about 830 nm, as delivered with laser apparatus including GaAlAs diodes as the laser energy source.

Thus, a method for the treatment of myocardial infarction in a subject in need of such treatment involves delivering a myocardial protective effective amount of light energy having a wavelength in the visible to near-infrared wavelength range to an ischemic area of the myocardium including and/or adjacent to an area of infarct, i.e. to myocardial cells in the ischemic zone. In preferred embodiments, delivering the myocardial protective amount of light energy includes selecting a surface power density of the light energy sufficient to deliver a predetermined power density of light energy to the area of the ischemic area of the myocardium. The power density to be delivered to the tissue is selected to be at least about 0.01 mW/cm², preferably about 10 mW/cm² or more. In one embodiment, the selected or predetermined power density is selected from the range of about 13 mW/cm² to about 150 mW/cm², including about 50 mW/cm² to about 90 mW/cm².

To deliver the desired power density at the level of the myocardial tissue, a relatively greater surface power density of the light energy is needed, and is calculated taking into account attenuation of the light energy as it travels from the skin surface through various tissues including skin, bone and fat tissue. Factors known to affect penetration and to be taken into account in the calculation include skin pigmentation, and the location of the affected myocardial area, particularly the depth of the area to be treated relative to the surface. For example, to obtain a power density of 50 mW/cm² in the myocardium at a depth of 3 cm below the skin surface may require a surface power density of 500 mW/cm². The large difference is a result of the high degree of scattering of light energy by the lungs and the absorption of energy by the ribs and sternum. The higher the level of skin pigmentation, the higher the required surface power density to deliver a predetermined power density of light energy to a subsurface myocardial site.

To treat a patient suffering from the effects of myocardial infarction, the light source is placed in contact with or immediately adjacent to a region of skin, for example on the chest, adjacent an ischemic area surrounding an infarcted area of the myocardium as may be identified using standard medical techniques including, but not limited to, electrocardiography, echocardiography or radionuclide testing. The power density calculation to determine how much power needs to be delivered at the surface preferably takes into account factors including skin coloration, distance to affected site in the myocardium, etc. that affect penetration and thus power density at the affected site, and the power used and the surface area treated are adjusted accordingly.

The precise power density selected for treating a patient depends on a number of factors, including the specific wavelength of light selected, the extent of the myocardial infarction and thus the extent of the ischemic zone, the clinical condition of the subject with particular regard to coronary artery disease or other conditions affecting cardiovascular health, and the like. Similarly, it should be understood that the power density of light energy to be delivered to the affected myocardial area may be adjusted to be combined with any other therapeutic agent or agents. The selected power density will again depend on a number of factors, as noted above, also including the particular therapeutic agent(s) employed.

### EXAMPLE 1

An in vitro experiment was conducted to demonstrate some effects of light therapy according to a preferred embodiment on human cardiomyocytes. Primary Human Cardiomyocyte cells (hCMC) were obtained through Cambrex (Baltimore, Md.), catalog # CC-7127. hCMC cells were received thawed, and proliferating in tissue culture flasks filled with medium (SmBM) catalog # CC-3182. These adherent cells were detached from the flasks using the Cambrex protocol and were plated into 96 well plates (white plastic with clear bottoms, Becton Dickinson, Franklin Lakes N.J.) at a density of 1000 cells/well in a volume of 100 microliters.

A Photo Dosing Assembly (PDA) was used to provide precisely metered doses of laser light to the hCMC cells in the 96 well plate. The PDA comprised a Nikon Diaphot inverted microscope (Nikon, Melville, N.Y.) with a LUDL motorized x,y,z stage (Ludl Electronic Products, Hawthorne, N.Y.). An 808 nm laser was routed into the rear epi-fluorescent port on the microscope using a custom designed adapter and a fiber optic cable. Diffusing lenses were mounted in the path of the beam to create a “speckled” pattern, which was intended to mimic in vivo conditions after a laser beam passed through human skin. The beam diverged to a 25 mm diameter circle when it reached the bottom of the 96 well plate. This dimension was chosen so that a cluster of four adjacent wells could be lased at the same time. Cells were plated in a pattern such that a total of 12 clusters could be lased per 96 well plate. Stage positioning was controlled by a Silicon Graphics workstation and laser timing was performed by hand using a digital timer. The measured power density passing through the plate for the hCMC cells was 90 mW/cm².

A CellTiter-Glo Luminescent Cell Viability Assay (Promega, Madison, Wis.) was used to measure the effects of 808 nm laser light on the hCMC cells. This assay generates a “glow-type” luminescent signal produced by a luciferase reaction with cellular ATP. The CellTiter-Glo reagent is added in an amount equal to the volume of media in the well and results in cell lysis followed by a sustained luminescent reaction that was measured using a Glo-Runner luminometer (Turner Biosystems, Sunnyvale, Calif.). Amounts of ATP present in the hCMC cells were quantified in Relative Luminescent Units (RLUs) by the luminometer.

The CellTiter-Glo assay was used to compare hCMC culture wells that had been lased with 90 mW/cm² at a wavelength of 808 nm with unlased control wells. Dosing time was 1 minute, resulting in a total energy dose of 5.4 Joules/cm². The CellTiter-Glo reagent was added 5 min after lasing completed and the plate was read 5 minutes later, after the cells had lysed and the luciferase reaction had stabilized. Twelve wells were lased and compared to an equal number of unlased control wells. The average RLUs measured for the control wells was 2329+/−116 while the laser group showed a 28% increase in ATP content to 2755+/−225. A student's unpaired t-test was performed on the data with a resulting p-value of 1×10⁻⁷, indicating that the increases in cellular ATP levels due to lasing were statistically significant.

An increase in cellular ATP concentration is related to cellular metabolism and is considered to be an indication that the cell is viable and healthy. These results are novel and significant in that they show the positive effects of laser irradiation on cellular metabolism in in vitro, primary, human, cardiomyocyte cell cultures.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for the treatment of myocardial infarction in a subject in need of such treatment, said method comprising delivering a myocardial protective effective amount of light energy having a wavelength in the visible to near-infrared wavelength range to an area of the myocardium of the subject that includes an area of infarct wherein delivering the myocardial protective effective amount of light energy comprises delivering a specified power density of light energy to the area of the myocardium.

2. A method in accordance with claim 1 wherein the predetermined power density is a power density of about 13 mW/cm².

3. A method in accordance with claim 1 wherein the predetermined power density is a power density selected from the range of about 0.1 mW/cm² to about 150 mW/cm².

4. A method in accordance with claim 3 wherein the predetermined power density is selected from the range of about 10 mW/cm² to about 100 mW/cm².

5. A method in accordance with claim 1 wherein the light energy has a wavelength of about 630 nm to about 904 nm.

6. A method in accordance with claim 5 wherein the light energy has a wavelength of about 780 nm to about 840 nm.

7. A method in accordance with claim 1 wherein delivering a myocardial protective effective amount of light energy to the area of the myocardial of the subject including the area of infarct comprises placing a light source in contact with a region of skin adjacent the area of the myocardium including the area of infarct.

8. A method in accordance with claim 1 wherein delivering a myocardial protective effective amount of light energy to the area of the myocardium of the subject including the area of infarct comprises determining a surface power density of the light energy sufficient to deliver the power density of light energy to the area of the myocardium.

9. A method in accordance with claim 8 wherein determining a surface power density comprises determining the surface power density of the light energy sufficient for the light energy to traverse the distance between the skin surface and the area of the myocardium.

10. A method in accordance with claim 9 wherein determining the surface power density further comprises. determining the surface power density sufficient to penetrate body tissue between the skin surface and the area of the myocardium.

11. A method in accordance with claim 1, wherein the light is delivered in pulses at a frequency of about 1 Hz to about 1 kHz.
